(19) United States
(12) Patent Application Publication
(10) Pub. No.: US 2003/0143208 A1
(43) Pub. Date: Jul. 31, 2003

(54) Oxidative stress-resistant cell lines and methods of use thereof

(76) Inventor: Haralambos Ischiropoulos, Media, PA (US)

Correspondence Address: DANN DORFMAN HERRELL & SKILLMAN, SUITE 720, 1601 MARKET STREET, PHILADELPHIA, PA, 19103-2307, US

(21) Appl. No.: 10/337,049

(22) Filed: Jan. 6, 2003

Related U.S. Application Data
(60) Provisional application No. 60/344,994, filed on Jan. 4, 2002.

(51) Int. Cl. A61K 45/00; C12N 5/08

(52) U.S. Cl. 424/93.7; 435/368

(57) ABSTRACT

Novel oxidative stress-resistant cell lines are provided. Such cell lines may be used to advantage in methods for the treatment neurodegenerative disorders associated with oxidative damage and subsequent neuronal cell loss, including, but not limited to, Parkinson's disease and head trauma.

### CROSS REFERENCES TO RELATED APPLICATIONS

This application claims priority under 35 U.S.C. §119(e) to U.S. Provisional Patent Application No. 60/344,994 filed on Jan. 4, 2002, the entire disclosure of which is incorporated by reference herein

### GOVERNMENT RIGHTS

Pursuant to 35 U.S.C. Section 202(c), it is acknowledged that the United States Government has certain rights in the invention described herein, which was made in part with funds from the National Institutes of Health Grant No. NIAAG13766.

### FIELD OF THE INVENTION

This invention relates to the development of cell lines and the use thereof in transplantation methods as a means to treat neurodegenerative disorders involving oxidative damage and subsequent neuronal cell loss. More specifically, oxidative stress-resistant cell lines are provided which may be used to advantage in transplantation methods to alleviate the deleterious affects of neuronal cell loss associated with oxidative injury or oxidative stress-induced apoptotic cell death.

### BACKGROUND OF THE INVENTION

Several publications are cited in this application by numerals in parentheses in order to more fully describe the state of the art to which this invention pertains. Full citations for these references are found at the end of the specification. Several patent documents are also cited in the application. The disclosure of each of these citations is incorporated by reference herein.

Parkinson's disease (PD) is a common neurodegenerative disorder affecting at least 500,000 people in the United States, with approximately 50,000 new cases reported annually. Clinically, the disease is characterized by a decrease in spontaneous movements, gait difficulty, postural instability, rigidity and tremor. The disorder appears to be slightly more common in men than women, and the average age of onset is approximately 60. Surprisingly, there is an increase in prevalence among people below 40. However, PD is still most prominent among people in their 70s and 80s.

To date, there is no cure for PD, but treatments are available which demonstrate variable efficacy. In many cases, patients are only mildly affected and need no treatment for several years after the initial diagnosis. However, when symptoms grow severe, the drug levodopa may be administered to help replenish dopamine levels in the brain. Unfortunately, long-term use of levodopa therapy causes complications such as dyskinesia, or uncontrolled movements.

In patients who are very severely affected by PD, a type of brain surgery known as pallidotomy has reportedly been effective in reducing the debilitating symptoms of the disease. Another form of brain surgery, involving transplantation of healthy fetal tissue into the brain of affected patients, is also being tested as a means to slow the progression of the disease.

The symptoms of PD are caused by the degeneration of the pigmented neurons in the substantia nigra of the brain which results in decreased dopamine availability. Previously, investigators hypothesized that the decline in dopamine level arose solely from the severe loss of dopaminergic neurons in the nigrostriatal pathway. However, recent studies indicate that the dopamine deficit in the affected regions of the brain significantly exceeds the loss of dopaminergic neurons, which suggests that dopamine synthesis may be impaired before cellular demise. Other studies using experimental models of PD also showed a reduction in dopamine metabolism-related markers, such as tyrosine hydroxylase and dopamine transporters, that is far greater than that attributable to the loss of neuronal cell bodies alone.

In addition, experimental evidence from studies of humans and other animals suggests that oxidative stress contributes to the pathogenesis of PD. Besides PD, oxidative injury has been implicated in the pathogenesis of other neurodegenerative disorders including Alzheimer's disease, dementia with Lewy bodies, amyotrophic lateral sclerosis and Huntington's disease. Also, high levels of nitrated alpha synuclein in “zones of cell death” have been found following experimental head trauma. It is believed that head trauma and the observed neuronal cell death which occurs over the ensuing days/weeks is due to the same sort of oxidative damage/apoptosis mechanism.

Oxidative injury occurs when the compensatory antioxidant capacity of cells is overwhelmed by excess production of reactive species that damage lipids, nucleic acids, proteins and other cellular components. Both reactive oxygen and nitrogen species are produced in vivo and may act synergistically to form nitrating agents that modify proteins as well as other biomolecules such as thiols, aldehydes and lipids. For example, superoxide reacts with nitric oxide to generate peroxynitrite. Biologically active nitrating agents are formed when peroxynitrite is in the presence of CO₂ or other catalysts, which then convert native tyrosine residues in proteins into 3-nitrotyrosine.

Studies using the 1-methyl-4-phenyl-1,2,3,6-tetrahydropyridine (MPTP) model of PD indicate that peroxynitrite may be a mediator of nigrostriatal damage in PD. The potential role of peroxynitrite in the pathogenesis of PD was further suggested when monoamine-producing PC12 cells were exposed to peroxynitrite which induced dose-dependent alterations in dopamine synthesis that could not be attributed to cell death or the oxidation of dopamine.

Tyrosine hydroxylase (TH) is one target of peroxynitrite. TH is a non-heme iron, tetrahydrobiopterin-dependent protein which catalyzes the conversion of tyrosine to L-dihydroxyphenylalanine (L-DOPA). TH catalytic activity is the rate-limiting step in the biosynthesis of catecholamines (1), and the loss of ability to synthesize catecholamines is an important step in the development of PD and other neurodegenerative diseases (2-6). Thus, an important contributor to the dopamine deficiency in PD may be the early loss of TH activity followed by a decline in TH protein levels in the brain.

TH is a selective target for nitration following administration of the parkinsonian toxin, MPTP, to mice and following exposure of PC12 cells to either peroxynitrite or 1-methyl-4-phenylpyridiniun ion (7). The nitration of TH tyrosine residue(s) is temporally associated with loss of enzymatic activity, and the magnitude of inactivation was proportional to the number of TH molecules that were nitrated in PC12 cells. For example, in the mouse striatum, the tyrosine nitration-mediated loss in TH activity paralleled the decline in dopamine levels, whereas the levels of TH protein remain unchanged for the first 6 hours post MPTP injection (7).

A recent report suggested that exposure of recombinantly purified TH to peroxynitrite in vitro results not only in nitration of tyrosine residues but also in the formation of covalently linked dimers and oxidation of cysteine residues (8). The same report also indicated that cysteine oxidation rather than tyrosine nitration is responsible for the loss of TH enzymatic activity (8). Cysteine, methionine, tryptophan and tyrosine appear to be the principal amino acids in proteins modified by peroxynitrite in vitro (9-14). To resolve the apparent differences, the reaction of peroxynitrite with recombinantly purified rat TH in vitro was re-examined and no evidence of cysteine oxidation was found. Oxidation of one cysteine residue per molecule of TH was observed only at high peroxynitrite concentrations, and three cysteine residues were oxidized in partially unfolded protein. In addition, amino acid analysis failed to show any alteration of methionine, tryptophan or any other amino acid residues.

Digestion and sequence analysis of TH peptides indicated that nitration of tyrosine 423 (tyr423) is the primary residue modified by peroxynitrite. The lack of nitration of a mutant TH, Tyr423Phe, in which Tyr423 was substituted with Phe423, further confirmed that tyr423 is the sole amino acid residue nitrated by peroxynitrite. In addition, no loss in TH enzymatic activity was detected after peroxynitrite treatment of the Tyr423Phe mutant. Stopped flow experiments further revealed reactivity with the ferrous iron in TH which is typical of metalloproteins reacting with peroxynitrite (15-17). Taken together, these data strongly implicate nitration of tyr423 as the causative event leading to TH inactivation by peroxynitrite.

### SUMMARY OF THE INVENTION

In accordance with the present invention, human oxidative stress-resistant cell lines are provided for the treatment of neurodegernative disorders associated with oxidative injury and neuronal cell death. These cell lines can be derived from ECV304, NT2N, PC12, PC2, MD-9, and human fibroblast cells by (1) seeding cells in culture under conditions that allow cells to undergo cell division, (2) contacting the cells with reactive oxygen and nitrogen species, (3) propagating the cells in the presence of the reactive oxygen and nitrogen species, and (4) isolating the cells that survive in the presence of the reactive oxygen and nitrogen species. In a preferred embodiment of the invention, these steps are repeated several times, preferably about 5 times, most preferably the steps are repeated at least ten times. The reactive oxygen and nitrogen species include, without limitation, peroxynitrite, hydrogen peroxide, rotenone, paraquat, dopamine or 1-methyl-4-phenyl-1,2,3,6-tetrahydropyridine.

In another embodiment of the invention, the oxidative stress-resistant cells derived as described above are transfected with a mutant tyrosine hydroxylase that has been modified such that tyr423 has been replaced with phenylalanine. As a result, the oxidative stress-resistant cells express a functional tyrosine hydroxylase that is resistant to the same oxidative stresses. In yet another embodiment, tyrosine residues 428 and 432 may also be modified to produce mutated tyrosine hydroxylase that is no longer nitrated at positions 428 and 432.

In a related aspect of the invention, methods are provided for treating mammals having neurological disease characterized by a dopamine deficiency. The methods involve transplanting therapeutically effective amounts of either the oxidative stress resistant cells or the oxidative stress-resistant cells transfected with mutant tyrosine hydroxylase into the brains of mammals to overcome the neurological deficits caused by dopamine deficiency.

In yet another aspect of the invention, methods are provided for treating Parkinson's disease. The methods involve transplanting therapeutically effective amounts of either the oxidative stress-resistant cells or the oxidative stress-resistant cells transfected with mutant tyrosine hydroxylase into the brains of patients with Parkinson's disease.

In yet another aspect of the invention, methods are provided for inhibiting the neuronal cell loss associated with head trauma in a patient. The methods involve transplanting therapeutically effective amounts of either the oxidative stress-resistant cells or the oxidative stress-resistant cells transfected with mutant tyrosine hydroxylase into the brains of patients suffering from head trauma.

### BRIEF DESCRIPTION OF THE DRAWINGS

FIG. 1 is a schematic diagram highlighting the effects of tyrosine nitration on tyrosine hydroxylase and dopamine production during the various stages of Parkinson's disease. Strategies for therapeutic intervention to treat the symptoms most common with each stage of the disease are also shown.

FIG. 2 is a diagram of the crystal structure of rat tyrosine hydroxylase. The sites of nitration are indicated at tyrosine residues 423 and 428 (tyrosine 432 is not shown).

FIGS. 3A-3C are graphs showing the effect of oxidative and nitrative stress on wild type and mutant tyrosine hydroxylase enzyme activity. FIG. 3A shows that the enzymatic activity of the wild-type, Tyr428Phe mutant and Tyr432Phe mutant tyrosine hydroxylase is inhibited by exposure to nitrating and oxidizing peroxynitrite whereas the enzymatic activity of the Tyr423Phe mutant was unaffected. FIG. 3B shows the ability of the Tyr423Phe mutant protein to resist inactivation by exposure to different concentrations of peroxynitrite as compare to wild type TH. FIG. 3C shows the ability of the Tyr423Phe mutant protein to resist inactivation by exposure to different concentrations of peroxynitrite as compare to wild type TH when subjected to the Lovenberg assay.

### DETAILED DESCRIPTION OF THE INVENTION

A fundamental and critical pathological finding in Parkinson's disease (PD) is a deficit in dopamine production due in part to the inactivation of tyrosine hydroxylase (TH) by oxidative stress as well as the premature death of a subset of neurons that generate dopamine. These neurons, which are also referred to as dopaminergic neurons, are mostly found in the substantia nigra of the brain. One remedy for treating the symptoms of PD is the supplementation of dopamine administered orally. However, this approach does not treat the disease and does not provide a long-term therapy as continuous use of dopamine results in down-regulation of its receptors as well as unwanted side-effects. Another approach for treating PD which has had moderate success entails transplantation of dopamine producing neurons and fetal cells into the brains of PD patients. The transplants are tolerated well, but unfortunately they provide only a temporary relief as presumably the same molecular mechanisms that kill the dopaminergic neurons in the first place also destroy the transplanted cells.

Preconditioning is a method used experimentally to adapt tissues to otherwise lethal conditions. For example, short periods of ischemia, that do not compromise the viability of heart or brain tissue, have been shown to protect the same tissues from subsequent lethal heart attacks or strokes. Previous studies have also revealed evidence of oxidative injury including nitrated proteins in the brains of patients suffering from PD.

It is known that other neuronal degenerative diseases involve oxidative injury and loss of neuronal cells. These include Alzheimer's disease, dementia with Lewy bodies, amyotrophic lateral sclerosis and Huntington's disease. Also, high levels of nitrated alpha synuclein in “zones of cell death” have been found following experimental head trauma. It is believed that head trauma and the observed neuronal cell death which occurs over the ensuing days/weeks is due to the same sort of oxidative damage/apoptosis mechanism.

Based on these findings, a more effective therapeutic strategy for the treatment of patients suffering from neurodegenerative disorders associated with neuronal cell loss has been developed. The inventive method entails introduction of preconditioned cells into the brains of such patients, the cells having been rendered resistant to the stresses that contribute to the eradication of dopamine and dopaminergic neurons. Thus, in accordance with the present invention, novel oxidative stress-resistant cell lines derived by culturing cells in the presence of lethal amounts of reactive oxygen and nitrogen species are provided. These cell lines may be used to advantage in therapeutic methods for the treatment of PD and other neurodegenerative disorders affected by oxidative injury or oxidative stress-induced apoptotic cell death.

In one embodiment of the invention, cells from the Human endothelial cell line, ECV304, have been preconditioned to lethal concentrations of hydrogen peroxide. This cell line is characterized by the up-regulation of antioxidant and protective cellular molecules as well as by the down-regulation of pro-death genes. As a result, the cell line remains viable even under intense oxidative stress conditions.

In accordance with another aspect of the invention, an alternative cell line is provided for use in therapeutic methods for the treatment of neurodegenerative disorders. Cells conditioned to be resistant to oxidative stress are transfected with a mutant TH gene. The mutant TH has been modified so that tyr423 is replaced with phenylalanine to eliminate nitration of the protein at that amino acid position. The cells so transfected may or may not produce dopamine. This approach provides a significant therapeutic advantage as the recombinantly expressed mutant TH retains its catalytic activity in the presence of oxidative stress inside cells that are preconditioned to the same stress.

I. Definitions:

The following definitions are provided to facilitate an understanding of the present invention:

“Dopaminergic neurons” refer to neurons which generate dopamine, the neurotransmitter molecule which transmits nerve signals from neuron to neuron.

“Oxidative stress” as used herein refers to toxic microenvironmental conditions created by the overproduction of reactive intermediates or “oxidants” generated as side products of oxygen and nitrogen metabolism.

“Nucleic acid” or a “nucleic acid molecule” as used herein refers to any DNA or RNA molecule, either single or double stranded and, if single stranded, the molecule of its complementary sequence in either linear or circular form. In discussing nucleic acid molecules, a sequence or structure of a particular nucleic acid molecule may be described herein according to the normal convention of providing the sequence in the 5′ to 3′ direction. With reference to nucleic acids of the invention, the term “isolated nucleic acid” is sometimes used. This term, when applied to DNA, refers to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism.

When applied to RNA, the term “isolated nucleic acid” refers primarily to an RNA molecule encoded by an isolated DNA molecule as defined above. Alternatively, the term may refer to an RNA molecule that has been sufficiently separated from other nucleic acids with which it would be associated in its natural state (i.e., in cells or tissues). An isolated nucleic acid (either DNA or RNA) may further represent a molecule produced directly by biological or synthetic means and separated from other components present during its production.

“Natural allelic variants”, “mutants” and “derivatives” of particular sequences of nucleic acids refer to nucleic acid sequences that are closely related to a particular sequence but which may possess, either naturally or by design, changes in sequence or structure. By closely related, it is meant that at least about 75%, but often, more than 90%, of the nucleotides of the sequence match over the defined length of the nucleic acid sequence referred to using a specific sequence identification number (SEQ ID NO). Changes or differences in nucleotide sequence between closely related nucleic acid sequences may represent nucleotide changes in the sequence that arise during the course of normal replication or duplication in nature of the particular nucleic acid sequence. Other changes may be specifically designed and introduced into the sequence for specific purposes, such as to change an amino acid codon or sequence in a regulatory region of the nucleic acid. Such specific changes may be made in vitro using a variety of mutagenesis techniques or produced in a host organism placed under particular selection conditions that induce or select for the changes. Such sequence variants generated specifically may be referred to as “mutants” or “derivatives” of the original sequence.

The present invention also includes active portions, fragments, derivatives and functional or non-functional mimetics of tyrosine hydroxylase polypeptides. An “active portion” of such a polypeptide means a peptide that is less than the full length polypeptide, but which retains measurable biological activity.

A “fragment” or “portion” of a tyrosine hydroxylase polypeptide means a stretch of amino acid residues of at least about five to seven contiguous amino acids, often at least about seven to nine contiguous amino acids, typically at least about nine to thirteen contiguous amino acids and, most preferably, at least about twenty to thirty or more contiguous amino acids. Fragments of the tyrosine hydroxylase polypeptide sequence, antigenic determinants, or epitopes are useful for eliciting immune responses to a portion of the tyrosine hydroxylase protein amino acid sequence.

Different “variants” of the tyrosine hydroxylase polypeptides exist in nature. These variants may be alleles characterized by differences in the nucleotide sequences of the gene coding for the protein, or may involve different RNA processing or post-translational modifications. The skilled person can produce variants having single or multiple amino acid substitutions, deletions, additions or replacements. These variants may include inter alia: (a) variants in which one or more amino acids residues are substituted with conservative or non-conservative amino acids, (b) variants in which one or more amino acids are added to the polypeptide, (c) variants in which one or more amino acids include a substituent group, and (d) variants in which the polypeptide is fused with another peptide or polypeptide such as a fusion partner, a protein tag or other chemical moiety, that may confer useful properties to the Tyrosine hydroxylase polypeptide, such as, for example, an epitope for an antibody, a polyhistidine sequence, a biotin moiety and the like.

The term “functional” as used herein implies that the nucleic or amino acid sequence is functional for the recited assay or purpose.

The phrase “consisting essentially of” when referring to a particular nucleotide or amino acid means a sequence having the properties of a given SEQ ID NO. For example, when used in reference to an amino acid sequence, the phrase includes the sequence per se and molecular modifications that would not affect the basic and novel characteristics of the sequence.

A “replicon” is any genetic element, for example, a plasmid, cosmid, bacmid, phage or virus, that is capable of replication largely under its own control. A replicon may be either RNA or DNA and may be single or double stranded.

A “vector” is a replicon, such as a plasmid, cosmid, bacmid, phage or virus, to which another genetic sequence or element (either DNA or RNA) may be attached so as to bring about the replication of the attached sequence or element.

An “expression operon” refers to a nucleic acid segment that may possess transcriptional and translational control sequences, such as promoters, enhancers, translational start signals (e.g., ATG or AUG codons), polyadenylation signals, terminators, and the like, and which facilitate the expression of a polypeptide coding sequence in a host cell or organism.

Polymerase chain reaction (PCR) has been described in U.S. Pat. Nos. 4,683,195, 4,800,195, and 4,965,188, the entire disclosures of which are incorporated by reference herein.

The terms “transform”, “transfect”, “transduce”, shall refer to any method or means by which a nucleic acid is introduced into a cell or host organism and may be used interchangeably to convey the same meaning. Such methods include, but are not limited to, transfection, electroporation, microinjection, PEG-fusion and the like.

The introduced nucleic acid may or may not be integrated (covalently linked) into nucleic acid of the recipient cell or organism. In mammalian cells, for example, the introduced nucleic acid may be maintained as an episomal element or independent replicon such as a plasmid. Alternatively, the introduced nucleic acid may become integrated into the nucleic acid of the recipient cell or organism and be stably maintained in that cell or organism and further passed on or inherited to progeny cells or organisms of the recipient cell or organism. Finally, the introduced nucleic acid may exist in the recipient cell or host organism only transiently.

Amino acid residues are identified in the present application according to the three-letter or one-letter abbreviations listed in Table I.

TABLE I:

| | 3-letter | 1-letter |
|---|---|---|
| Amino Acid | Abbreviation | Abbreviation |
| L-Alanine | Ala | A |
| L-Arginine | Arg | R |
| L-Asparagine | Asn | N |
| L-Aspartic Acid | Asp | D |
| L-Cysteine | Cys | C |
| L-Glutamine | Gln | Q |
| L-Glutamic Acid | Glu | E |
| Glycine | Gly | G |
| L-Histidine | His | H |
| L-Isoleucine | Ile | I |
| L-Leucine | Leu | L |
| L-Methionine | Met | M |
| L-Phenylalanine | Phe | F |
| L-Proline | Pro | P |
| L-Serine | Ser | S |
| L-Threonine | Thr | T |
| L-Tryptophan | Trp | W |
| L-Tyrosine | Tyr | Y |
| L-Valine | Val | V |
| L-Lysine | Lys | K |

Amino acid residues described herein are preferred to be in the “L” isomeric form. However, residues in the “D” isomeric form may be substituted for any L-amino acid residue, provided the desired properties of the polypeptide are retained. All amino-acid residue sequences represented herein conform to the conventional left-to-right amino-terminus to carboxy-terminus orientation.

The term “isolated protein” or “isolated and purified protein” is sometimes used herein. This term refers primarily to a protein produced by expression of an isolated nucleic acid molecule of the invention. Alternatively, this term may refer to a protein that has been sufficiently separated from other proteins with which it would naturally be associated, so as to exist in “substantially pure” form. “Isolated” is not meant to exclude artificial or synthetic mixtures with other compounds or materials, or the presence of impurities that do not interfere with the fundamental activity, and that may be present, for example, due to incomplete purification, addition of stabilizers, or compounding into, for example, immunogenic preparations or pharmaceutically acceptable preparations.

The term “substantially pure” refers to a preparation comprising at least 50-60% by weight of a given material (e.g., nucleic acid, oligonucleotide, protein, etc.). More preferably, the preparation comprises at least 75% by weight, and most preferably 90-95% by weight of the given compound. Purity is measured by methods appropriate for the given compound (e.g. chromatographic methods, agarose or polyacrylamide gel electrophoresis, HPLC analysis, and the like).

“Mature protein” or “mature polypeptide” shall mean a polypeptide possessing the sequence of the polypeptide after any processing events that normally occur to the polypeptide during the course of its genesis, such as proteolytic processing from a polyprotein precursor. In designating the sequence or boundaries of a mature protein, the first amino of the mature protein sequence is designated as amino acid residue 1. As used herein, any amino acid residues associated with a mature protein not naturally found associated with that protein that precedes amino acid 1 are designated amino acid −1, −2, −3 and so on. For recombinant expression systems, a methionine initiator codon is often utilized for purposes of efficient translation. This methionine residue in the resulting polypeptide, as used herein, would be positioned at −1 relative to the mature tyrosine hydroxylase protein sequence.

A “clone” or “clonal cell population” is a population of cells derived from a single cell or common ancestor by mitosis.

A “cell line” is a clone of a primary cell or cell population that is capable of stable growth in vitro for many generations.

II. Cell Lines:

In a specific embodiment of the invention, cell lines are derived from the ECV304 cell line (ATCC NO. 1998), a spontaneously transformed line of human endothelial-like cells isolated from umbilical cord.

In yet another embodiment of the invention, cell lines can be derived from NT2N, PC12 (ATCC Number: CRL-1721), PC2, MD-9 and human fibroblast cells. The NT2N cell line is derived from an embryonal carcinoma cell line (NTera-2 or NT2 cells (ATCC No. 1973)) which is transfectable and capable of differentiating into post-mitotic neuron-like cells (NT2N cells) following treatment with retinoic acid (27). NT2N cells have previously been used for cell transplantation to treat stroke patients by re-establishing neuronal networks in the brain. During the process of making NT2N cells resistant to oxidative stress (after approximately 5 cycles), the cells begin to differentiate into neuron-like cells without mitotic inhibitors or retinoic acid. In this semi-differentiated state, the cells are more resistant to oxidants.

PC12 cells are a widely used neuron-like clonal line derived from rat pheochromocytoma cells. PC12 cells have been successfully transplanted in the brains of PD patients. PC12 cells also show signs of differentiation and appear to respond robustly to growth factors like neuronal growth factor, NGF, as compared to untreated cells. PC2 cells are clonal variants of PC12. However, unlike PC12 and NT2N cells, PC2 cells do not produce dopamine.

Autologous human fibroblast cells and cell lines may also be rendered resistant to oxidative stress. Accordingly, the use of autologous fibroblast cell which may optionally be immortalized is also within the scope of the invention. However, the cell lines of the invention need not be autologous in origin. Unlike transplantation at other body sites, introduction of the heterologous cells into the brain does not require the concomitant administration of strong anti-rejection drugs as an immune response to the transplanted cells is not observed.

III. Exemplary Oxidative-Stress Resistant Cell Lines:

An oxidative stress-resistant cell line derived from ECV304 cells has been generated in accordance with the present invention. ECV304 cells were exposed to low concentrations of lethal reactive oxygen and nitrogen species. Suitable reactive oxygen and nitrogen species include, but are not limited to, peroxynitrite, hydrogen peroxide, rotenone, paraquat, dopamine and 1-methyl-4-phenyl-1,2,3,6-tetrahydropyridine. Cells that survived multiple cycles of exposure to these lethal oxygen and nitrogen species were then able to survive in the presence of 0.5 M hydrogen peroxide, which normally kills any cell within a few hours. Further propagation of the surviving cells will give rise to the novel oxidative stress-resistant cells of the present invention. Also contemplated within the scope of the present invention are cell lines derived from a single cell that survived the multiple cycles of exposure to the lethal oxygen and nitrogen species. Gene array and proteomic analyses revealed that the oxidative stress-resistant cells demonstrated increased levels of antioxidants and protective cellular molecules. Additionally, expression levels of pro-death genes were down-regulated in these cells.

Another oxidative stress-resistant cell line has been generated in which the oxidative stress-resistant cells described above are transfected with nucleic acids encoding a mutant TH, Tyr423Phe. These preconditioned cells express functional TH that is resistant to the same oxidative stresses.

IV. Uses of the Oxidative Stress-Resistant Cell Lines:

Methods employing the oxidative stress-resistant cells of the present invention provide a superior cell-based therapy for the treatment of neurodegenerative disorders that result from oxidative damage and the subsequent neuronal cell loss, including without limitation, head trauma and PD. The cell lines have been rendered resistant to the lethal effects of oxidative stress and thereby improve upon existing cell transplantation therapies for treating PD. The cells of the present invention will survive under conditions that normally kill dopaminergic cells in the PD patient and thus, may be transplanted to replace those cells that normally produce dopamine in the brain.

In another embodiment of the invention, the oxidative stress-resistant cells will be further fortified to protect against oxidative stresses by transfecting these cells with a mutant TH which retains its catalytic activity but is no longer a target for tyrosine nitration. The resulting preconditioned/transfected cells provide a significant therapeutic advantage by producing a mutant TH which retains its catalytic activity in the presence of oxidative stress.

In yet another embodiment of the invention, the oxidative stress-resistant cells may be used to advantage to further study the molecular and biochemical basis of preconditioning. The elucidation of the molecular basis of preconditioning will facilitate the development of novel therapeutic agents, such as immuno- and chemo-reagents, for the treatment of PD and other neurodegenerative disorders as well as facilitate the development of novel therapies to prevent heart attacks or strokes in patients in need thereof.

V. Transplantation of Oxidative Stress-Resistant Cells:

The present invention provides methods of treating a host with PD or another neurodegenerative disorder such as Alzheimer's disease, dementia with Lewy bodies, amylotrophic lateral sclerosis and Huntington's disease, by implanting the oxidative stress-resistant cells of the invention to the brains of patients in need thereof. Additionally, the oxidative stress-resistant cells may be transplanted into the brains of patients suffering head trauma, which may mitigate the subsequent neuronal cell death which ensues after the initial trauma event. As used herein, “treating a host” includes prophylactic, palliative and curative intervention in a disease process. The host may be any warm blooded mammal, such as humans, non-human primates, rodents and the like.

The cells of the invention will be prepared for implantation by suspending the cells in a physiologically compatible carrier, such as cell culture medium (e.g., Dulbecco's Modified Eagle Medium containing 10% fetal bovine serum) or phosphate buffered saline.

The volume of cell suspension to be implanted will vary depending on the site of implantation, and cell density in solution. In a preferred embodiment of the invention, a cell suspension of 10,000 to 25,000 oxidative stress-resistant cells will be administered in each injection. Several injections may be used in each host. Persons of skill in the art will be able to determine proper cell dosages for this purpose.

In another embodiment of the invention, tissue fragments or “patches” of oxidative stress-resistant cells may be implanted into the brains of PD patients. Exemplary tissue fragments will be 100 to 200 μm in diameter.

The cells of the invention may also be encapsulated by membranes prior to implantation. The encapsulation provides a barrier to the host's immune system and inhibits graft rejection and inflammation. Several methods of cell encapsulation are known in the art such as those described in U.S. Pat. Nos.: 4,353,888, 4,744,933, 4,749,620, 4,814,274, 5,084,350 or 5,089,272, each of which is incorporated by reference herein.

The cells of the invention will be administered to the hosts by surgical implantation or grafting into the brain. Suitable methods for transplantation of cells into the brains of patients having neurological disorders are provided in U.S. Pat. Nos.: 5,869,463 and 5,690,927, the entire disclosures of each being incorporated by reference herein. In an exemplary method of the invention, the oxidative stress-resistant cells may be grafted within the putamen and/or caudate nucleus. An exemplary method of cell transplantation has been described previously by Freed et al. (18). This method involves affixing a sterotactic ring to the skull and using magnetic resonance imaging to establish coordinates for four needle passes in the axial plane of the putamen. Two needle tracks are created in each side of the brain above the frontal sinus, one about 7 mm higher than the other. Four twist-drill holes through the frontal bone may be made along the planned axis of the tracks. The oxidative stress-resistant cells are implanted using a stainless-steel guide cannula with a graduated outer diameter of 1.5 to 0.6 mm. A rounded stylet is placed in the bore of the cannula during its passage to the posterior tip of the putamen. The stylet is then replaced with a needle containing transplant cells in a volume of approximately 20 μl, which is deposited continuously as the needle is withdrawn through the putamen. Two minutes must pass for stabilization of pressure and then the cannula is removed from the brain.

The transplant procedure may be performed while the host is awake, with local anaesthesia administered to the skin of the forehead. This will permit surgeons to assess the host's ability to speak and to move during the transplant procedure.

After the surgery, the host may optionally be administered immunosupressive drugs to prevent rejection of the implanted cells.

Further details regarding the practice of this invention are set forth in the following examples, which are provided for illustrative purposes only and are in no way intended to limit the invention.

### EXAMPLE I

### Preparation of Peroxynitrite-Resistant Cell Lines

Dopamine-producing cells preconditioned to the lethal conditions of peroxynitrite were prepared using the following protocol:

Human endothelial cells, ECV304, were cultured to 75% confluency in Dulbecco's Modified Eagle Medium (DMEM) containing 10% fetal bovine serum (FBS) and 1% penicillin/streptomycin. The cells were trypsonized, washed with DMEM/FBS, and then spun in a centrifuge at 450 g for 5 minutes in 15 ml Falcone tubes. The supernatant was discarded and the cell pellets were resuspended in PBS solution (Gibco). The cells in PBS solution were spun at 450 g for 5 minutes and the pellets were resuspended in 10 ml of treatment buffer (30 mM Na₂HPO₄, 96 mM NaCl, 5 mM KCl, 0.8 mM MgCl₂, 1 mM CaCl₂, 5 mM glucose; adjusted to pH 7.4 and sterilized).

The cell suspension was then treated with 1 mM peroxynitrite given via injections of 4 bolus to the side of the Falcone tubes. The tubes were rocked gently between injections to ensure mixing. Five minutes post-treatment, the cells were spun at 450 g for 5 minutes and resuspended in DMEM/FBS and plated. The cells were propagated until they reached 75% confluence. The treatment procedure was then repeated. After 6 to 8 treatment cycles, cells show nearly 50% survival after additional peroxynitrite treatment. These cells were isolated, cloned and designated as the peroxynitrite-resistant cell line.

This protocol is readily adaptable and may also be used to create oxidative stress-resistant cell lines derived from NTN2, PC12, PC2, MD-9, and human fibroblast cells.

### EXAMPLE II

### The Tyrosine Hydroxylase Activity of the Tyr423Phe Mutant is Unaffected by Exposure to Peroxynitrite

Inactivation of tyrosine hydroxylase (TH) has been observed in early stages of PD as well as the mouse MPTP model of PD (4). In the MPTP model, previous data revealed that TH is specifically modified by nitration of tyrosine residues (6), specifically tyrosine 423 (tyr423) and to a lesser extent tyrosine 428 and 432. A temporal association between the number of TH molecules modified and loss of activity was observed (7). In addition, amino acid analysis and fluorescence spectrometry of purified TH failed to detect any other amino acid modifications after nitration of TH.

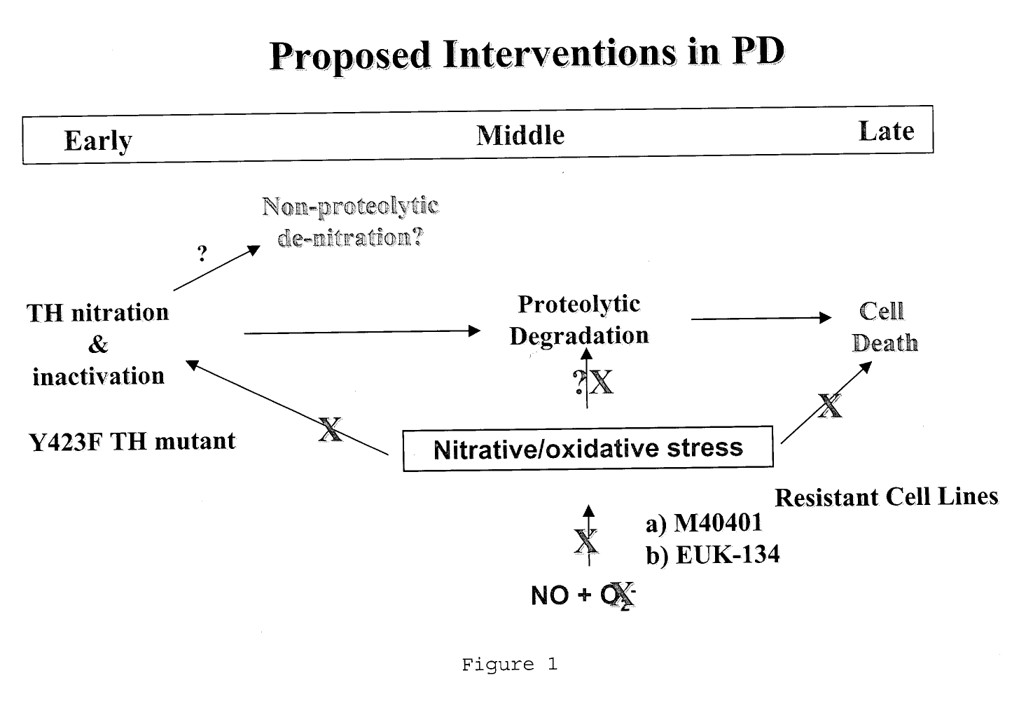

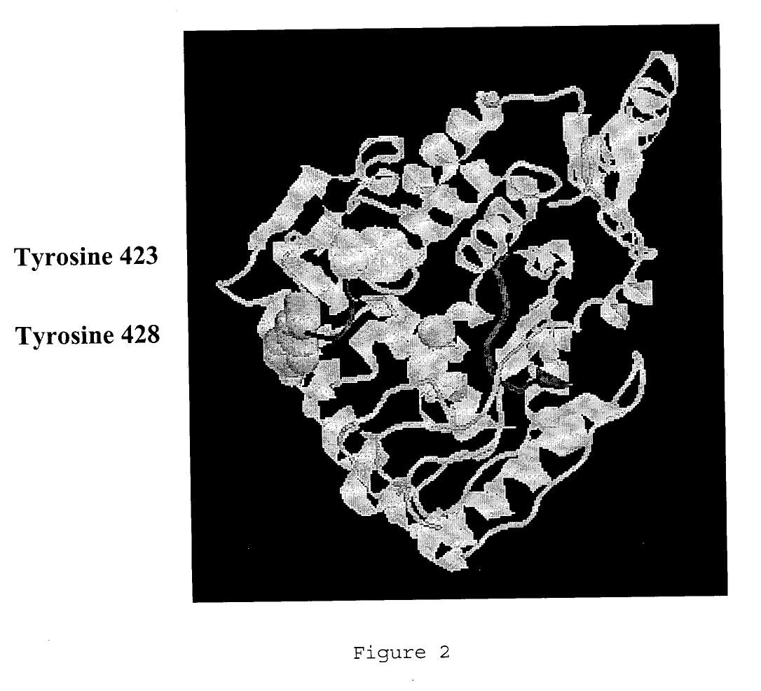

In PD patients, distinct changes in TH activity and concentration have also been described. During the early stage of the disease, a decrease in dopamine levels is apparent without a loss of either TH immunoreactivity or dopaminergic neurons (See FIG. 1). The middle stage of the disease is characterized by a loss of dopamine and immunoreactive TH without a significant loss of dopaminergic neurons. However, during the late stage of the disease, there is a loss of dopamine, TH and dopaminergic neurons.

Biochemical mechanisms which explain the changes in TH during the progression of PD have not yet been fully elucidated. However, published data suggest that during the initial stage of PD, nitrated TH causes the decrease in dopamine production, and in the middle stage of the disease, it is believed that nitration of TH induces a more rapid, (faster than normal), removal of the TH protein by proteosomes. However, in the late stage of the disease, it is believed that neuronal cell death causes the loss of dopamine, TH and dopaminergic neurons.

Efforts to limit the formation of nitrating agents by limiting the production of nitric oxide and superoxide have been successful in protecting mice and baboons from MPTP-induced neuronal cell death (19-21). Recently, Pong et al. showed that EUK134, a superoxide dismutase and catalase mimetic, prevented nitration of TH in cultured dopaminergic neurons after MPP⁺ challenge (22). Therefore, development of therapeutic agents that can prevent protein nitration without interfering with normal neuronal function may provide a means of limiting neuronal injury in PD patients.

Expression of Rat Mutant TH

To further confirm the site of nitration in vivo, the tyrosine residues 423, 428, and 432 were individually mutated to Phe. The Tyr423Phe, Tyr428Phe, and Tyr432Phe mutants were created with the QuikChange™ Site-Directed mutagenesis kit from Stratagene as previously described (25). Wild-type rat TH cDNA (GenBank accession no.: NM-012740) was subcloned into the mammalian pcDNA3.1+ expression vector (invitrogen) and used as the DNA template (25 ng) to create the mutants. Complete DNA sequencing data verified the presence of the appropriate mutation in the coding sequences of all recombinant proteins. The primers used to generate Tyr423Phe mutant are provided in Table II.

TABLE II
PCR Primers

| | |
|---|---|
| TH Y423F | (SEQ ID NO: 1) |

Sense: 5′-GCA GCT GTG CAG CCC TTC CAA GAT CAA ACC
TAC C-3′;

(SEQ ID NO: 2)

Antisense: 5′-G GTA GGT TTG ATC TTG GAA GGG CTG CAC
AGC TGC-3′;

The bold nucleotides highlight the mutated codons for amino acid 423. Complete DNA sequencing was performed by the DNA Sequencing and Gene Analysis Facility of the Molecular Genetics Program (Wake Forest University School of Medicine) using a Perkin Elmer/Applied Biosystems 377 Prism automated DNA sequencer. Complete DNA sequencing was performed to verify the presence of the appropriate mutation in the coding sequences of all recombinant proteins. This also established that the non-PCR-based mutagenesis (Tyr423Phe) did not introduce extraneous mutations. The Tyr428phe and Tyr432phe mutants were generated in a comparable fashion using the appropriate primer sets.

Transient Transfection of PC2 Cells

The mutant TH (Y423F-TH, Y428F-TH, and Y432F-TH) plasmids expressed in the pcDNA3.1 mammalian vector were grown in competent E. coli and plasmid DNA was purified using the Quiagen plasmid midi kit (Quiagen). PC2 cells were grown in 6-well plates with a density of 0.5×106 cells per well and plasmid DNA was transfected into PC2 cells using Lipofectamine 2000 (Invitrogen) in a serum free optimem medium (Invitrogen) for 5 hours. Transfection medium was supplemented by Dulbecco's modified Eagle's medium containing final concentration of 10% heat inactivated horse serum and 5% fetal bovine serum, and cells were allowed to grow for 48 hours at 37° C.

Purification and Activity Assay of wt- and Mutant-TH

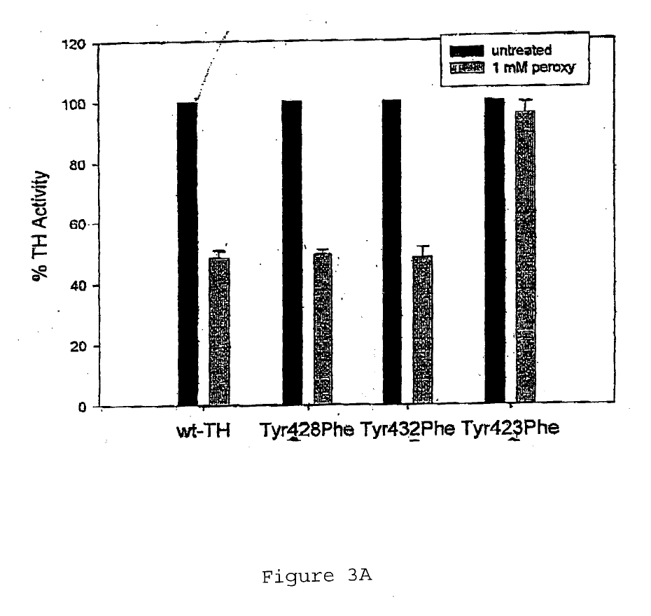

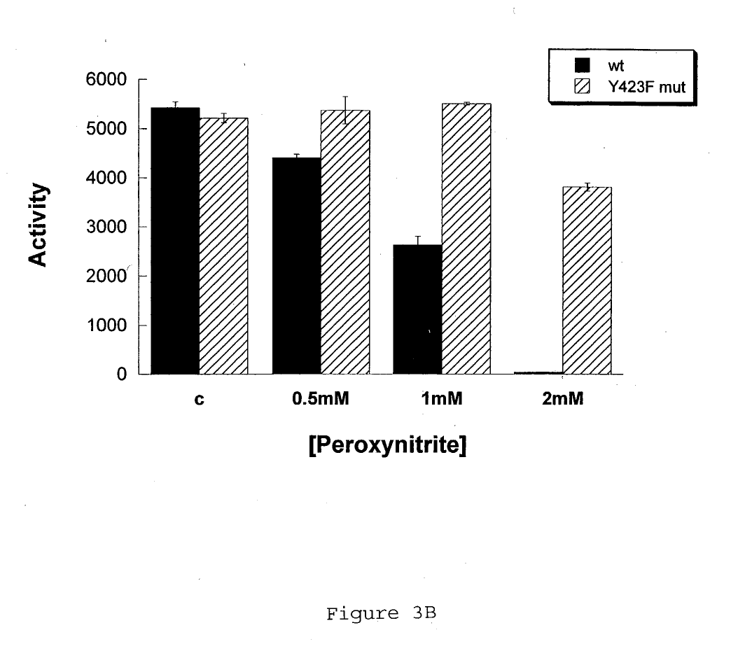

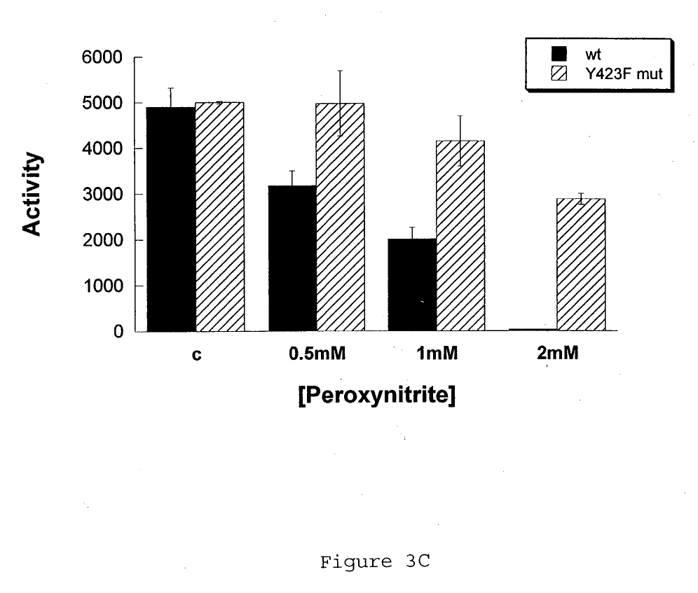

Recombinant wt- and mutant TH were isolated and purified from BL21(DE3)pLysS E. coli expressing the full-length wt-TH, Y423F-TH, Y428F-TH, or Y432F-TH cDNAs. Following isolation enzyme activity was assayed after treatment with peroxynitrite as previously described (25 and 26). Briefly, wt- or mutant-TH were exposed to several concentrations of peroxynitrite under identical conditions, in phosphate buffer 0.1 M containing DTPA 0.1 mM at pH 7.4. The activity of TH was assayed by the release of [³H]H₂O from [³H]tyrosine in the presence of catalase. The specific activity of the partially purified Tyr423Phe mutant TH was 20% of the wild type TH. It is also shown that the enzyme activity of the wild type, and the Tyr428Phe and Tyr432Phe mutants were inhibited by exposure to nitrating and oxidizing concentrations of peroxynitrite (FIG. 3A). However, no significant loss of enzymatic activity was observed with the Tyr423 Phe mutant. The ability of the Tyr423Phe mutant protein to resist inactivation by oxidative and nitrative stress as compared to wild type TH was further evaluated by exposing cell lysates to different peroxynitrite concentrations. Enzymatic activity was assessed using a standard assay and the Lovenberg assay as described in (26) (FIGS. 3B and 3C). The above results confirm that the modification of this residue by nitration provides the biochemical basis for the inactivation of the protein.

### EXAMPLE III

### Preparation of Oxidative Stress-Resistant Cells Transfected with Mutant Tyrosine Hydroxylase

As described in Example I, novel cell lines have been created where cells have been preconditioned to the lethal effects of peroxynitrite. These cell lines may be further modified to protect against the lethal effects of protein nitration.

Mutant Tyr423Phe TH as described in Example II, which retains TH catalytic activity, but is no longer nitrated at Tyr423 may be used to advantage to create a dual prong approach for the treatment of neuronal oxidative damage associated with PD and head trauma for example.

Human TH is encoded by a single gene wherein alternative splicing produces 4 variant mRNA types which have insertion or deletion of the 12 bp and 81 bp sequences from a single primary transcript. The primary tyrosine residue for nitration in human TH is Tyr423 of human TH3 (GenBank Accession No. Y00414-1) and Tyr 418 or Tyr427 of human TH4 (GenBank Accession No. M17589-1). The Try423 of human TH3 or Tyr418 or Tyr427 of human TH4 may be replaced by another amino acid, such as, phenylalanine as described in the previous examples.

The oxidative stress-resistant cells described in Example I may be then transfected with a mutant human TH to create preconditioned cells carrying functional TH which is resistant to the same stresses. Such cells may then be transplanted into the brains of patients with PD or head trauma to arrest or diminish the progression of the disease.

Mutant tyrosine hydroxylase may be transfected into the oxidative stress-resistant cells as described above, or via the use of retroviral vectors. TH cDNA will be inserted into HindIII-Cla-1 sites of a Maloney murine leukemia virus-derived plasmid, pLNCX, downstream of the CMV promoter. The pLNCX contains the gene for aminoglycoside phosphotransferase (neo) which is used as a selection marker. The DNA sequence encoding the mutant TH will be operably linked to the transcriptional promoter and a transcriptional terminator such as those commonly known in the art. The DNA sequence may also be linked to a transcriptional enhancer. Preferably, expression of the DNA in the preconditioned cells is constitutive. However, in alternative embodiments, mutant TH expression may be regulated with an inducible promoter. A variety of suitable inducible expression vectors having the characteristics noted above may be used to carry the DNA for transfection of the oxidative stress-resistant cells, and are known to those skilled in the art. Promoters for this purpose include without limitation, the metallothionine promoter, heat shock protein 70 promoter and glucocortocoid promoter. Such cells may then be used to advantage for the treatment of neurodegenerative disorders associated with oxidative injury and neuronal cell loss.

### References

1. Kaufman, S. (1995) Adv. Enzymol. Relat. Areas Mol. Biol. 70, 103-220.

2. Calne, D. B., and Langston, J. W. (1983) Lancet 2, 1457-1459.

3. MacGeer, P. L., Itagaki, S., Akiyama, H., and McGeer, E. G. (1989) Ann. Neurol. 24, 574-576.

4. Nagatsu, T. (1990) Neurochem. Res. 15, 425-429.

5. Olanow, C. W., Jenner, P., and Beal, M. F. (1998) Ann. Neurol. (suppl.) 44, S1-S196.

6. Fahn, S., and Przedborski, S. (2000) In: Merritt's neurology, edited by L. P. Rowland, New York:Lippincott Williams & Wilkins, p. 679-693.

7. Ara, J., Przedborski, S., Naini, A. B., Jackson-Lewis, V., Trifiletti, R. R., Horwitz, J., and Ischiropoulos, H. (1998) Proc. Natl. Acad. Sci. USA 95, 7659-7663.

8. Kuhn, D. M., Aretha, C. W., and Geddes, T. J. (1999) J. Neurosci. 19, 10289-10294.

9. Radi, R., Beckman, J. S., Bush, K. M., and Freeman, B. A. (1991) J. Biol. Chem. 266, 4244-4250.

10. Pryor, W. A., Jin, X., and Squadrito, G. L. (1994) Proc. Natl. Acad. Sci. USA 91, 11173-11177.

11. Ischiropoulos, H., and Al-Mehdi, A. B. (1995) FEBS Lett. 364, 279-282.

12. Alvarez, B., Rubbo, H., Kirk, M., Barnes, S., Freeman, B. A., and Radi, R. (1996) Chem. Res. Toxicol. 9, 390-396.

13. Alvarez, B., Ferrer-Sueta, G., Freeman, B. A., and Radi, R. (1999) J. Biol. Chem. 274, 842-848.

14. Beckman, J. S., Ischiropoulos, H., Zhu, L., van der Woerd, M., Smith, C., Chen, J., Harrison, J., Martin, J. C., and Tsai, M. (1992) Arch. Biochem. Biophys. 298, 438-445.

15. Radi, R. (1996) Chem. Res. Toxicol. 9, 828-835.

16. Cassina, A. M., Hodara, R., Souza, J. M., Thomson, L., Castro, L., Ischiropoulos, H., Freeman, B. A., and Radi, R. (2000) J. Biol. Chem. 275, 21409-21415.

17. Minetti, M., Pietraforte, D., Carbone, V., Salzano, A. M., Scorza, G., and Marino, G. (2000) Biochemistry 39, 6689-6697.

18. Freed C. R., Greene, P. E., Breeze, R. E., Tsai, W. -Y., DuMouchel, W., Kao, R., Dillon, S., Winfield, H., Culver, S., Trojanowski, J. Q., Eidelberg, D., and Fahn, S. (2001) N. Engl. J. Med. 344:710-719.

19. Ferrante, R. J., Hantraye, P., Brouillet, E., and Beal, M. F. (1999) Brain Res. 823, 177-182.

20. Przedborski, S., Kostic, V., Jackson-Lewis, V., Naini, A. B., Simonetti, S., Fahn, S., Epstein, C. J., and Cadet, J. L. (1992) J. Neuroscience 12, 1658-1667.

21. Przedborski, S., Jackson-Lewis, V., Yokoyama, R., Shibata, T., Dawson, V. L., Dawson, T. M. (1996) Proc. Natl. Acad. Sci. USA 93, 4565-4571.

22. Pong, K., Doctrow, S. R., and Baudry, M. (2000) Brain Res. 881, 182-189.

23. Yohrling, I. V. G. J., Mockus, S. M., and Vrana, K. E. (1999) J. Mol. Neurosci. 12, 23-34.

24. Vrana, K. E., Rucker, P., and Kumer, S. C. (1994). Life Sci. 55, 1045-1052.

25. Blanchard-Fillion, B. et al, (2001). J. Biol. Chem. 276:46017-46023.

26. Bensinger, R. E., Klein, D. C., Weller, J. L. and Lovenberg, W. M. (1974) Radiometric assay of total trytophan hydroxylation by intact cultured pineal glands. Journal of Neurochemistry. 23: 111-117.

27. Pleasure S. J. (1992) Pure, postmitotic, polarized human neurons derived from NTera 2 cells provide a system for expressing exogenous proteins in terminally differentiated neurons. J. Neurosci. 12(5) :1802-1815.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. An oxidative stress-resistant cell line.

2. The oxidative stress-resistant cell line is a human oxidative stress-resistant cell line.

3. A method for obtaining an oxidative stress-resistant cell line, comprising the steps of:
a) seeding cells in culture for a suitable time period to allow said cells to undergo at least one round of cell division;
b) contacting said cells with at least one reactive oxygen and nitrogen species;
c) propagating said cells in the presence of said reactive oxygen and nitrogen species; and
d) isolating said cells that survive in the presence of said reactive oxygen and nitrogen species.

4. The method of claim 3, wherein steps a)-d) are repeated at least once.

5. The method of claim 3, wherein steps a)-d) are repeated at least ten cycles.

6. The method of claim 3, further comprising clonally selecting said isolated cells.

7. The method of claim 3, wherein said cells are selected from the group consisting of ECV304, MD-9, NT2N, PC12, PC2 and human fibroblast cells.

8. The method of claim 3, wherein said reactive oxygen and nitrogen species is selected from the group consisting of peroxynitrite, hydrogen peroxide, rotenone, paraquat dopamine, and 1-methyl-4-phenyl-1,2,3,6-tetrahydropyridine.

9. The human oxidative stress-resistant cell line of claim 2, wherein said cells are transfected with a mutant tyrosine hydroxylase in which the primary tyrosine residue for nitration has been mutated.

10. The cell line of claim 9, wherein said mutant tyrosine hydroxylase has been modified such that the primary tyrosine residue for nitration has been replaced with phenylalanine.

11. The cell line of claim 10 wherein the tyrosine at pos 423 has been replaced with phenylalanine.

12. A method for treating a mammal having a neurodegenerative disorder that results from oxidative damage and results in neuronal cell loss, comprising transplanting into said mammal a therapeutically effective amount of the cells of claim 1.

13. A method for treating a host having Parkinson's disease comprising implanting into the putamen of said host a therapeutically effective amount of the cells of claim 1.

14. A method for treating a mammal having a neurodegenerative disorder that results from oxidative damage causing subsequent neuronal cell loss, comprising transplanting into said mammal a therapeutically effective amount of the cells of claim 9.

15. A method for treating a host having Parkinson's disease comprising implanting into the putamen of said host a therapeutically effective amount of the cells of claim 9.

16. A method for treating head trauma having a neurodegenerative disorder that results from oxidative damage causing subsequent neuronal cell loss, comprising transplanting into said mammal a therapeutically effective amount of the cells of claim 9.

17. A method for treating a host having head trauma comprising implanting into the putamen of said host a therapeutically effective amount of the cells of claim 9.


SEQUENCE LISTING (2 sequences)

SEQ ID NO: 1 - DNA, 34 bp, Artificial Sequence
  Primer
gcagctgtgcagcccttccaagatcaaacctacc

SEQ ID NO: 2 - DNA, 34 bp, Artificial Sequence
  Primer
ggtaggtttgatcttggaagggctgcacagctgc